(19) United States
(12) Patent Application Publication
(10) Pub. No.: US 2026/0034363 A1
(43) Pub. Date: Feb. 5, 2026

(54) Neural Stimulation Device Monitoring Evoked Signals

(71) Applicant: Wisconsin Alumni Research Foundation, Madison, WI (US)

(72) Inventors: Kip Ludwig, Middleton, WI (US); James Trevathan, Madison, WI (US); Rex Chin-Hao Chen, Waukesha, WI (US); Brandon Coventry, Verona, WI (US)

(21) Appl. No.: 18/793,478

(22) Filed: Aug. 2, 2024

(51) Int. Cl. A61N 1/36

(52) U.S. Cl. CPC A61N 1/36139; A61N 1/36062

(57) ABSTRACT

The ability to more precisely locate a region of spinal stimulation through tracking of evoked signals at multiple displaced electrodes allows improved display, and adjustment of the stimulation location and/or power. Stimulation location uncertainty can be reduced by information extracted from different arrival times at the displaced electrodes instead of estimated based on the physical location of the stimulation electrode. The deduced determination of regions of spinal stimulation for different signals allows for the rejection of non-neural signals.

### STATEMENT REGARDING FEDERALLY SPONSORED RESEARCH OR DEVELOPMENT

This invention was made with government support under OD025340 awarded by the National Institutes of Health. The government has certain rights in the invention.

### CROSS-REFERENCE TO RELATED APPLICATIONS

--

### BACKGROUND OF THE INVENTION

The present invention relates to an apparatus for neural stimulation and in particular to an apparatus providing improved control of such stimulation by monitoring evoked neural signals at multiple electrodes.

Neuromodulation provides a precise electrical stimulation of neural pathways useful in treating drug-resistant neurological and psychiatric disorders. This technique's efficacy is sensitive to the magnitude and location of neural activation which may involve considerable trial and error. For example, in spinal cord stimulation, trial electrodes may be placed within the spine and various stimulation combinations explored in consultation with the patient. After a trial period, a more permanent implantation may occur during which adjustments may still be necessary. The neurophysiological mechanisms of this technique are not completely understood.

Evoked compound action potential (eCAP) techniques measure evoked potentials at monitoring electrodes displaced from one or more stimulating electrodes. The present inventors have previously developed an eCAP technique for determining an effective stimulation point, a location which cannot be determined simply from the location of the stimulus electrodes. This eCAP technique uses multiple monitoring electrodes and exploits measures of time delay between pairs of electrodes to deduce neural signal velocity. This velocity information is used to back propagate the nerve signals to the effective stimulation point.

### SUMMARY OF THE INVENTION

The present inventors recognized that the ability to identify an effective stimulation points using a set of remote electrodes allows improved artifact identification. This improved artifact identification can be used to provide an improved display and adjustment of the effective stimulation point and/or can be used for automatic adjustment of the stimulation over time, for example, to compensate for electrode movement or the like.

Knowledge of the effective stimulation point (as opposed to the physical location of the stimulating electrodes) provides not only precise knowledge about the location of the stimulation but can also distinguish between different effective stimulation points for different nerve fiber types allowing closed loop or interactive adjustment of the effective stimulation point for a particular set of nerve fibers either by electrode selection or stimulation power.

In one embodiment, the invention provides a neural stimulation device having inputs for receiving signals from implantable electrodes positionable along a nerve and having a stimulation power source communicating the electrodes producing a nerve stimulation signal. A stimulation control circuit controls the stimulation power source to selectively apply stimulation to at least one stimulation electrode and communicates with other electrodes for monitoring electrical activity signals displaced from the stimulation position. The stimulation control circuit may further operate to distinguish artifact signals in the electrical activity signals unrelated to the stimulation based from nerve signals related to the stimulation based on imputed location of an effective stimulation points individually consistent with the artifacts and nerve signals. Using the identified artifacts signals, the stimulation control circuit may display the effective stimulation points for the nerve signals distinguished from effective stimulation points of the artifacts signals.

It is thus a feature of at least one embodiment of the invention to greatly improve the adjustment of electrodes used for neural stimulation by providing a display of effective stimulation points distinguished from artifacts.

The stimulation control circuit may display multiple effective stimulation points associated with different nerve fiber types.

It is thus a feature of at least one embodiment of the invention to allow a clinician better understanding of nerve stimulation distinguishing among fiber types.

In one embodiment, the neural stimulation device may include a model of the nerve having tunable model parameters and may use the distinguished artifacts signals and nerve signals to adjust the model parameters. The stimulation control circuit may then receive an input describing a desired effective stimulation point for the electrical activity signals and apply the input to the model to adjust the stimulation circuit to move the effective stimulation point according to the input.

It is thus a feature of at least one embodiment of the invention eliminate or reduce trial and error necessary to adjust the stimulation point on a patient by providing a model which can be used in the optimization process

In another embodiment, the invention provides a neural stimulation device providing a set of implantable electrodes positionable along a nerve. A stimulation power source is controlled by a stimulation control circuit to apply stimulation to one or more stimulation electrodes having a stimulation power, position, and a time and to monitor electrical activity signals at multiple monitoring electrodes displaced from the stimulation position. The stimulation control circuit operates to deduce an effective stimulation point of the stimulation to modify at least one of the power and position of the stimulation electrodes based on the electrical activity signals at the multiple monitoring electrodes.

It is thus a feature of at least one embodiment of the invention to make use of an effective stimulation point, rather than physical electrode position, to provide improved accuracy to neuromodulation by a stimulating device.

The neural stimulation device may deduce effective stimulation points from timing differences between corresponding electrical activity signals between multiple monitoring electrodes.

It is thus a feature of at least one embodiment of the invention to provide a system for determining an effective stimulation point independent of location of the stimulating electrode or an understanding of the precise interaction between the stimulating electrode and the nerve. Generally, the location of the stimulation is a function not only of the electrode position but also electrode power and nerve physiology and will generally not be centered within the electrical field produced by the electrode and can move during the stimulation.

The neural stimulation device may further identify a set of different effective stimulation points for different types of nerve fibers based on deduced nerve signal velocities.

It is thus a feature of at least one embodiment of the invention to permit control of neural stimulation specific to particular nerve fiber types.

The stimulation control circuit may use the determined effective stimulation point to reject non-neural signals in modifying the power and/or position of the stimulation electrode, for example, by rejecting non-neural signals having effective stimulation points beyond the stimulating electrode position.

It is thus a feature of at least one embodiment of the invention to address problems of signal artifacts which can interfere with the measurement of evoked signals for control of neuromodulation in a clinical setting.

The stimulation control circuit may receive a desired stimulation position and adjust the at least one power and position of the stimulation electrode to move the effective stimulation point to reduce the separation between the desired stimulation position and the effective stimulation point.

It is thus a feature of at least one embodiment of the invention to permit dynamic control of the stimulation position through the use of effective stimulation point determination.

The stimulation control circuit may work with a model of the nerve and fit data of the stimulation power, position, and time and the electrical activity signals to the model to determine the effective stimulation point.

It is thus a feature of at least one embodiment of the invention to constrain the present technique with a physiological model, for example, using recorded waveforms and arrival timing to improve model parameters, which are then used to produce an effective stimulus location more precisely than simple geometric back propagation.”

The model may alternatively provide a set of modeled electrical activity signals excluding non-neural signals to provide the effective simulation location based on the modeled electrical activity signals.

It is thus a feature of at least one embodiment of the invention to use the model to exclude artifacts inconsistent with a neural system.

These particular objects and advantages may apply to only some embodiments falling within the claims and thus do not define the scope of the invention.

### BRIEF DESCRIPTION OF THE DRAWINGS

FIG. 1 is a fragmentary elevational view in phantom of a patient's spine and an implanted neural stimulator suitable for use with the present invention as placed for spinal stimulation showing in separate insets, the neural stimulator electrodes and monitoring electrodes in the epidural space of the vertebrae near the spinal nerve;

FIG. 2 is an enlarged view of the neurostimulator electrodes aligned with time plots of the evoked signals at various monitoring electrodes showing a geometric extrapolation to effective stimulation points for various nerve fibers;

FIG. 3 is a block diagram depicting a fitting of the measurements of the monitoring electrodes to a physiological model for improved artifact rejection and/or calculation of effective stimulation points;

FIG. 4 is a flowchart of the program executable by the neural stimulator of FIG. 1;

FIG. 5 is a depiction of a screen display such as may display output for the neurostimulator of FIG. 1 and accept user input; and

FIG. 6 is a perspective fragmentary view of a nerve with a self adjusting permeable electrode cuff to minimize measurement artifacts.

### DETAILED DESCRIPTION OF THE INVENTION

### Background

The process of nerve activation, leading to the generation of evoked compound action potentials (eCAPs), is fundamental in neuromodulation. When a nerve fiber is electrically stimulated, it undergoes a localized change in transmembrane potential, which triggers an action potential. This action potential propagates along the axon through the sequential opening of voltage-gated ion channels, resulting in a cascade of depolarization and repolarization.

The activating function is a key element in the study of electrical neuromodulation. This function indicates the change in transmembrane potential due to an external electric field and is mathematically defined as the second spatial derivative of the electric field along the nerve fiber. The peak of this function signifies a significant change in membrane potential, denoting potential initiation sites for action potentials along the nerve fiber. The region where this peak occurs, known as the “recruited volume,” encompasses the area where nerve fibers are sufficiently depolarized to initiate action potentials. Factors such as electrode configuration, stimulus intensity and duration, and nerve tissue properties, including fiber diameter, myelination, and internodal distance, determine the spatial extent of the electric field and the volume of nerve tissue that reaches the activation threshold.

eCAPs, or evoked compound action potentials, represent the collective electrical signals generated by a group of nerve fibers in response to stimulation. They are important for assessing the efficacy and spatial precision of neuromodulation, as they reflect the induced neural activity by the external electric field. Interestingly, eCAPs predominantly originate from the edges of the recruited volume where action potentials can transmit effectively.

On the contrary, within the recruited volume, the propagation of eCAPs is significantly affected by the phenomenon of collision block. This occurs when oppositely propagating action potentials, originating from different points along the nerve fiber, intersect and nullify each other. Primarily taking place in the central regions of the recruited volume, this collision block leads to the peripheral origination of eCAPs that contribute to the recorded neural response.

Thus, the activation origin of eCAPs is more precisely defined as the point within the recruited volume from which these compound nerve signals can propagate without encountering collision block. This activation origin is referred as an effective stimulation point in this patent. From this origin, eCAPs start their outward journey, moving away from the center of stimulation and towards the recording electrodes. This activation origin is a critical factor in the dynamics of nerve activation and propagation under electrical stimulation and cannot be precisely determined by the location of the stimulating electrode alone.

### Spinal Stimulation Embodiment

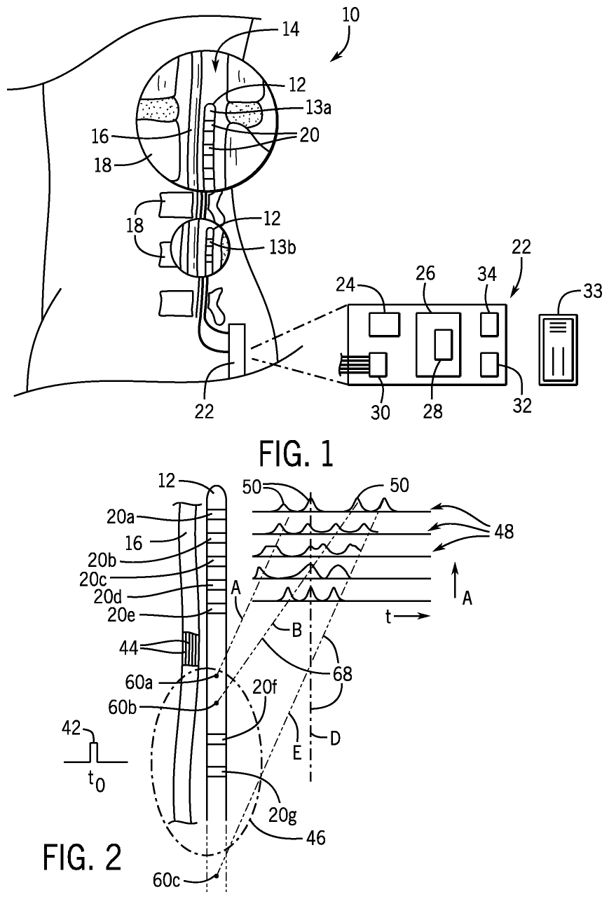

Referring now to FIG. 1, a neural stimulator 10 in one embodiment may provide for an elongate electrode assembly 12 that can be threaded into the epidural space 14 of a patient's spine within the vertebrae 18 adjacent to the spinal nerve 16. In one embodiment the electrode assembly 12 may include two physically independent support structures 13a holding monitoring electrodes and 13b holding stimulation electrodes, to reduce the effect of electrode migration, or a single support structure may hold both sets of electrodes.

In either case, the electrode assembly 12 may provide for an insulating tubular body supporting on its outer surface, for example, multiple ring-shaped electrodes 20 extending circumferentially around the tubular body. Generally, the monitoring electrodes will include electrodes 20 separated at a regular fixed and known spacing along the length of the electrode assembly 12. These electrodes may be a linear array as depicted or a two-dimensional or three-dimensional array which may be readily adapted to the techniques discussed below. The stimulating electrodes may include one or typically two electrodes 20 or may preferably provide multiple such electrodes 20 to provide flexibility in changing the location of the stimulation.

Referring also to FIG. 2, more specifically, in one example, electrodes 20 may include stimulating electrodes 20f-20g for stimulating the nerve fibers of the nerve 16 and monitoring electrodes 20a-20e for receiving signals from the evoked potentials transmitted along nerve 16.

Each electrode 20 is independently connected by internal wiring to a stimulation unit 22 which may, for example, be implanted within tissue beneath the skin. The stimulation unit 22 may provide, for example, a processor 24 communicating with electronic memory 26 holding a stored program 28 as will be discussed below. The processor 24 may further communicate with interface electronics 30 that may synthesize an output stimulation signal directed at the stimulation electrodes 20f and 20g of the electrode assembly 12 and may monitor received signals from the monitoring electrodes 20a-20e and digitize those signals for processing by the processor 24 executing the stored program 28. An internal rechargeable battery 32 may provide power to the stimulation unit 22 which may also provide a wireless transceiver 34 for programming the stimulation unit 22 or outputting data from the stimulation unit 22 to a programming device 33 such as a tablet or cell phone operated by the user or a technician.

Referring now to FIGS. 2 and 4, in operation, and as indicated by process block 40, the program 28 of the stimulation unit 22 may apply a stimulating pulse 42 at time t₀ to stimulating electrodes 20f and 20g. Generally, the stimulating pulse will be as short as practical and much shorter than that used during actual therapy in order to better distinguish nerve signal timing. For example, this pulse may be on the order of 20 μs and typically less than 100 μs. Commonly used pulse widths in spinal cord therapy range between 100 and 500 μs, while those for vagus nerve therapy typically range between 250 and 500 μs. In this regard, the stimulating pulse 42 may be controlled with respect to voltage level, polarity, timing, and shape so as to promote stimulation of nerve fibers 44 within the nerve 16. During operation, the electrodes 20f and 20g may produce an electrical field 46 defining a recruited volume within which stimulation will occur. Generally, however, the exact point of stimulation will be highly influenced by the surrounding tissue and the current state and physiology of the nerve fibers 44 and thus may not be accurately determined simply by the placement of the electrodes 20f and 20g.

The nerve signals evoked by the electrical field 46 will travel (vertically as depicted in FIG. 2) along the nerve to monitoring electrodes 20a-20e where they will induce received waveforms 48 for each monitoring electrode 20 whose timing and shape may be recorded by the stimulation unit 22. Generally the received waveforms 48 for each monitoring electrode 20 will be a composite of nerve pulses 50 from multiple different nerve fibers having different propagation velocities. The received waveforms 48 will also include signals that are not from the nerve 16 henceforth termed artifact electrical signals, for example, from muscle activity, motion artifact, environmental electrical interference, and the like.

A simple calculation of the velocity of the nerve pulses 50, by measuring the distance between the stimulating electrodes 20e and 20f and any given monitoring electrode 20a-20e and the time lapse between time t₀ and the timing of the given nerve pulses 50 in the received waveforms 48, is hampered by lack of knowledge of the precise point of electrical stimulation in each of the fibers by the stimulating electrodes 20f and 20g (as discussed above) and the difficulty of isolating and identifying the given nerve pulses 50 in the waveforms 48.

In one embodiment, these difficulties may be addressed by using a delay and sum (beamforming) algorithm per process block 62. In this technique, multiple point by point additions of each of the received waveforms 48 is performed with the received waveforms shifted with respect to each other by a slowly incrementing delay amount, the delay amount representing an expected delay induced phase shift between the received waveforms 48 for neural signals propagating in the nerve 16 for a set of different velocities. The result is a summed waveform for each of a range of different velocities. The amplitude of peaks in those summed waveforms coupled with the implicit velocity associated with the summed waveforms, and knowledge of the registration of the summed waveforms with respect to t₀, allows a back propagation of these amplitude values to locations on the nerve 16. Repeating this process for each of the summed waveforms while changing an imputed velocity produces a composite signal origin waveform along the nerve 16 allowing identification of a set of effective stimulation points 60 as points of local maximum.

Per process block 70, the location of the effective stimulation points 60 and the timing of their associated neural pulses 50 (also identified as local maxima within the summed waveforms) define signal velocities associated with each stimulation point 60 represented as the slope of trajectory lines 68 in FIG. 2.

It is contemplated that other methods of making use of the multiple nerve activities signals to better localize the effective stimulation point 60 and to determine the associated velocities may be used including methods of decomposing the waveforms 48 into pulses 50, for example, using correlations, wavelet decompositions, and the like with subsequent geometric extrapolation.

In this example, four different trajectory lines 68 are depicted related to the first four pulses 50 of each of the waveforms 48 labeled A-D and associated with a different one of electrodes 20a-20e. Each of these trajectory lines 68 may map to a different stimulation point 60 labeled 60a-60d corresponding to the trajectory lines 68 per program 28 of process block 70.

Each of these stimulation points 60a-60d may further be mapped to a nerve type based on the implicit velocity represented by the slope of the trajectory lines 68. Historically, nerve fibers have been classified according to their nerve signal velocities, dictated by myelin thickness, axon diameter, and internodal distance, into categories such as A (a, 3, 6, 7), B and C fibers. In this representation, the highest velocity trajectory line 68, labeled line A, and the second highest velocity of trajectory line 68, labeled B, map respectively to stimulation points 60a and 60b, both locations being positioned between a midpoint between the stimulating electrodes 20f and 20g and the monitoring electrodes 20a-20e and thus representing valid neural signals.

Generally, simulation points 60 beyond the monitoring electrodes 20a-20e by more than a predetermined distance, for example 1 to 2 cm, may be considered to be invalid. By way of example, trajectory line 68, labeled E, is associated with a point 60c removed on the far side of the stimulating electrodes 20f and 20g from the monitoring electrodes 20a and 20e and trajectory line 68, labeled D, which fails to converge on the nerve (in this case, representing an infinite velocity) and can be identified as non-neural signals, for example, being electromyographic signals or typically, in the case of line D, external electrical interference. The imputed stimulation points 60 can thus be used to identify and remove artifacts signals for process block 72 of program 28 (shown in FIG. 4), a process that can be understood geometrically as tracing backward on the trajectory line 68 to pulses 50 and removing those pulses 50 from the waveforms 48. Artifact signals may also be identified by velocities outside the range of plausible neural velocities.

Sometimes, EMG signals have a waveform shape that changes as it reaches the recording electrodes, providing the appearance of a high velocity signal when analyzed by the passage across electrodes 20a-20e and thus a velocity within the range of nerve fibers. Nevertheless, the stimulation 0.60 would still point back to an area that is far from the active electrodes 20f and 20g showing the benefits of this analysis approach.

In summary, the stimulation unit 22 may use the determined effective stimulation point to reject non-neural signals in modifying the power and/or position of the stimulation electrode. For example, by rejecting non-neural signals having effective stimulation points in non-biologically plausible locations, such as instances where low stimulation amplitude results in activation centimeters away from the active electrodes, or effective stimulation points appearing several centimeters away from the active electrode on the opposite side of the active electrodes when propagating from the side of the monitoring electrodes, albeit this is also not the location of where insulation ends. Biologically plausible locations include threshold activation at the center of cathode and for anodic activation, it starts around the area near the anode (virtual cathode). and it moves away from the its starting point when power are increased. After removal of the artifacts, the cleaned waveforms 48 may then again be processed to refine stimulation points 60 in an iterative process.

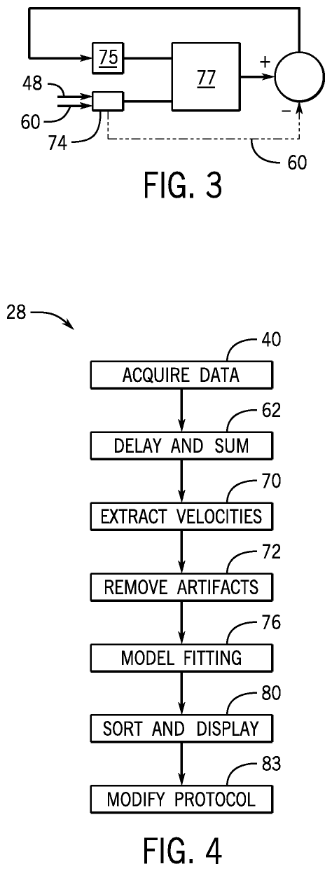

Referring now to FIG. 3 in an alternative embodiment, the cleaned waveforms 48 may be provided as parameter inputs 74 to an electrical/physiological model 77, for example, a finite element model of a nerve with multiple nerve fiber types, for example, as described in US patent 8,812,26 entitled System and Method to Define Target Volume for Stimulation of the Spinal Cord and Peripheral Nerves, hereby incorporated by reference. This electrical/physical model, making use of predefined parameter 75, may then be used to improve the adjustment of the stimulation point as will be described below, as well as to back propagate the neural waveforms 48 to points 60 using a more sophisticated nerve model that does not assume constant nerve velocities per process block 76. The reason to perform back propagation in these models is to ensure that the effective stimulation points generated by the model match the recorded effective stimulation points. If there is a discrepancy, the model is adjusted accordingly. The determined effective stimulation point 60 may be provided to the electrical/physical model 77 and used to iteratively refine the parameter 75 to provide a match between the effective stimulation point computed from the recordings and the effective stimulation point generated from the electrical/physical model. This fitting process can be used to eliminate any discrepancy between the nerve model and the true patient nerve anatomy in relation to the stimulation electrodes, thereby increasing the predictive power of these models to closely reflect the patient's nerve response.

Optionally, as indicated by process block 80, the points 60 and identified nerve fibers may, for example, be displayed to a user or technician, for example, in graphical form as represented by FIG. 2 or in a tabular form as wirelessly communicated with the external programming device 33 (shown in FIG. 1) or the like by the stimulation unit 22 for the purpose of configuring the stimulating stimulation unit 22 or for clinical study.

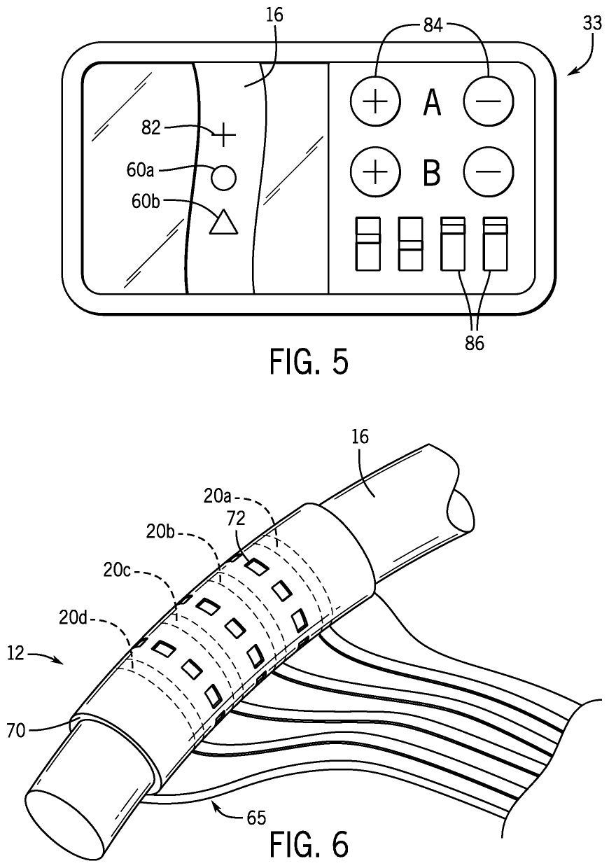

Referring now to FIG. 5, the programming device 33 (shown in FIG. 1) may provide information to a healthcare professional for adjusting a stimulation points 60. In one embodiment, a schematic representation of the nerve 16 is depicted together with stimulation points 60a and 60b identified to different fiber types (e.g. A, B) based on signal velocity discussed above and after removal of artifacts as described above. The healthcare professional may then identify a desired new location for stimulation for a particular nerve fiber type by maneuvering a cursor 82 to that desired location using, for example, displacement buttons 84 associated with the fiber type. As noted above, the stimulation points 60 are not a simple function of the location of the stimulating electrodes 20f and 20g. In one embodiment, the healthcare professional may make various adjustments using adjustment sliders 86 to change location of the stimulation (when multiple stimulating electrodes 20 are provided), polarity, wave shape, duration and amplitude. Largely this would be a trial and error process. Preferably however, the previously developed model per process block 76 may be used to automatically optimize these various values of the adjustments sliders 86 to achieve a close fit of an actual stimulation point 60 to the desired location indicated by the cursor 82, for example, using known optimization techniques such as gradient descent, simulated annealing, etc. In this way, the healthcare professional can provide faster, reproducible and clinically grounded adjustment of the stimulation points 60.

Per process block 83, the identified stimulation points 60 may then be used to provide a closed loop control of the stimulation, for example, receiving a desired stimulation location or a stimulation location adjustment through wireless communication at a time of configuration (as discussed above) and then afterwards during the monitoring the actual stimulation point 60 with respect to the nerve 16 during periodic eCAP operations. This actual stimulation location with respect to the nerve 16 may, for example, be determined from knowledge of the location of the monitoring electrodes 20a-20e under the assumption that they have not migrated or shifted possible because of their physical separation with the stimulating electrodes 20f and 20g. Alternatively, non-neural signals previously characterized as artifacts that are related to fixed physical structures in the body such as muscles or the like may be used to derive an absolute reference position with respect to the body of the stimulation point.

Referring momentarily to FIG. 2, in this latter approach, artifacts E related to, for example, an electromyographic signal may be distinguished from artifact D which does not have converge within a reasonable distance of the region of the electric field 46 and thus likely represents interference outside of the body or too far away from the region of the electric field 46 to provide a useful reference. The stimulation point 60c may then be monitored for a period of time with respect to stability and used for the purpose of automatically adjusting the stimulation locations 60a and 60b with respect to the nerve 16 instead of or in conjunction with the location of the monitoring electrodes 20a-20e.

After the actual and desired stimulation locations are determined, a difference between the desired stimulation location and the simulation point 60 can be assessed and this difference used in a closed loop fashion, for example, according to a model or a hill climb technique to change any of the amplitude, duration or shape of the stimulating pulse 42 and/or to select among different stimulating electrodes 20 to better track a particular location for consistent clinical result. Generally higher amplitude change the stimulation point 60 by increasing the recruitment volume.

After this adjustment, the duration of the stimulus is returned to clinical duration levels which will typically be longer than the stimulus used for assessing stimulation point 60 which will desirably be short for better time resolution.

Referring now to FIG. 6, variations in a fluid layer between the nerve 16 and the individual electrodes 20 have been determined to create artifacts in the arrival time measurement of the nerve pulses 50 believed to be caused by a mismatch between the velocity of electrical signals in the nerve 16 versus the isolated fluid layer and/or changes in the ionic environment of the nerve 16 caused by the isolation of this fluid layer. Accordingly, in one embodiment, an electrode assembly 12 may be provided to minimize a fluid layer between the electrodes 20 and the nerve 16 through a self adjusting nerve cuff 65 providing an elastically biased substrate 70 tending to curl tightly around the nerve 16, for example, of an insulating polymer material, this biasing thus excluding excess fluid. In addition or alternatively, openings 72 may be placed between each electrode 20a-20d to promote the interchange of ions between any fluid layer between the substrate 70 and nerve 16 and fluid outside of the nerve cuff 65, or an ion or fluid permeable material may be employed for the substrate 70. To the extent that this isolated fluid layer causes a spreading of the apparent electrical pulses as a superposition of a combination of faster propagating and slower propagating signals, computational techniques may also be used to compensate for these artifacts, for example deconvolution empirically determined time delay adjustments or the like.

Although the example of the spine stimulator has been discussed, it will be appreciated that this system and these principles can be applied to other types of neural stimulation including, for example, vagus nerve stimulation, Hypoglossal Nerve Stimulation (HNS) for Sleep Apnea Treatment, and the like.

In addition, the stimulation of the nerve need not be electrical but may include various methods that can possibly activate neurons, such as chemical, mechanical, optical stimulation, ultrasound stimulation, magnetic stimulation, optogenetics, and sonogenetics.

For recording and imaging, it is not limited to electrical recording but includes any other possible ways of recording neural signals, such as ultrasound imaging, photoacoustic imaging, optical imaging (voltage-sensitive imaging, calcium-sensitive imaging), and magnetoelectrical imaging (fMRI).

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “an electronic computer” and “a processor” or “the microprocessor” and “the processor,” can be understood to include one or more of these devices that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

References to “a processor” should be understood to include electronic computers, microprocessors, microcontrollers, FPGA devices, ASIC devices and similar programmable or program defined electronic circuits and collections of such devices that can communicate in a stand-alone and/or a distributed environment(s) and can thus be configured to communicate via wired or wireless communications with other processors. Furthermore, references to memory, unless otherwise specified, can include one or more processor-readable and accessible memory elements and/or components that can be internal to the processor or external to the processor and accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

We claim:

1. A neural stimulation device comprising:
inputs for receiving signals from implantable electrodes positionable along a nerve;
a stimulation power source communicating with at least implantable electrodes and producing a nerve stimulation signal;
a stimulation control circuit controlling the stimulation power source to selectively apply stimulation to at least one stimulation electrode selected from the implantable electrodes at a stimulation power, a position, and a time and communicating with electrodes of the implantable electrodes other than the at least one stimulating electrode for monitoring electrical activity signals at multiple monitoring electrodes selected from the implantable electrodes and displaced from the stimulation position; and
the stimulation control circuit operating to distinguish artifact signals in the electrical activity signals unrelated to the stimulation based from nerve signals in the electrical activity signals related to the stimulation based on imputed location of an effective stimulation points individually consistent with the artifact signals and nerve signals;
wherein the stimulation control circuit uses the identified artifacts signals to display the effective stimulation points for the nerve signals distinguished from effective stimulation points of the artifacts signals.

2. The neural stimulation device of claim 1 wherein the stimulation control circuit displays only effective stimulation points of the nerve signals.

3. The neural stimulation device of claim 1 wherein the stimulation control circuit displays multiple effective stimulation points associated with different nerve fiber types.

4. The neural stimulation device of claim 1 including a model of the nerve having model parameters; and
wherein the stimulation control circuit uses the distinguished artifacts signals and nerve signals to adjust the model parameters; and
wherein stimulation control circuit receives an input describing a desired effective stimulation point for the electrical activity signals and applies the input to the model to adjust the stimulation circuit to move the effective stimulation point according to the input.

5. The neural stimulation device of claim 4 wherein the model is a finite element model.

6. A neural stimulation device comprising:
a set of implantable electrodes positionable along a nerve;
a stimulation power source communicating with at least one electrode of the set of implantable electrodes and producing a nerve stimulation signal;
a stimulation control circuit controlling the stimulation power source to selectively apply stimulation to at least one stimulation electrode selected from the implantable electrodes at a stimulation power, a position, and a time and communicating with electrodes of the set of implantable electrodes other than the at least one stimulating electrode for monitoring electrical activity signals at multiple monitoring electrodes selected from the implantable electrodes and displaced from the stimulation position; and
the stimulation control circuit operating to deduce an effective stimulation point of the stimulation to modify at least one of the power of the stimulation and position of the stimulation electrode based on the electrical activity signals at the multiple monitoring electrodes.

7. The neural stimulation device of claim 6 at least one electrode of the set of implantable electrodes producing the nerve stimulation signal is physically independent of the electrodes of the set of implantable electrodes other than the at least one stimulating electrode.

8. The neural stimulation device of claim 6 wherein the stimulation control circuit uses the effective stimulation point to identify non-neural signals and uses at least one effective stimulation point of a non-neural signal to monitor and correct for movement of the electrodes of the set of implantable electrodes other than the at least one stimulating electrode with respect to a patient.

9. The neural stimulation device of claim 6 wherein the stimulation control circuit deduces the effective stimulation point from timing differences between corresponding electrical activity signals between multiple monitoring electrodes.

10. The neural stimulation device of claim 6 wherein the stimulation control circuit further identifies a set of different effective stimulation points for different types of nerve fibers based on deduced nerve signal velocities.

11. The neural stimulation device of claim 6 wherein the stimulation control circuit uses the effective stimulation point to reject non-neural signals in modifying the at least one power and position of the at least one stimulation electrode with respect to the monitoring electrodes.

12. The neural stimulation device of claim 11 wherein the stimulation control circuit rejects non-neural signals having effective stimulation points beyond a position of the stimulating electrode.

13. The neural stimulation device of claim 6 wherein the stimulation control circuit receives a desired stimulation position and adjusts the at least one power and position of the stimulation electrode to move the effective stimulation point to reduce a separation between the desired stimulation position and the effective stimulation point.

14. The neural stimulation device of claim 6 wherein the stimulation control circuit provides a model of the nerve and fits data of the stimulation power, position, and time and the electrical activity signals to the model to determine the effective stimulation point.

15. The neural stimulation device of claim 14 wherein the model provides a set of modeled electrical activity signals excluding non-neural signals to provide the effective simulation location based on the modeled electrical activity signals.

16. The neural stimulation device of claim 15 wherein the model is a finite element model of the nerve.

17. A method of adjusting neural stimulation using a monitor device providing:
inputs for receiving signals from implantable electrodes positionable along a nerve;
a stimulation power source communicating with at least implantable electrodes and producing a nerve stimulation signal;
a stimulation control circuit controlling the stimulation power source to selectively apply stimulation to at least one stimulation electrode selected from the implantable electrodes at a stimulation power, a position, and a time and communicating with electrodes of the implantable electrodes other than the at least one stimulating electrode for monitoring electrical activity signals at multiple monitoring electrodes selected from the implantable electrodes and displaced from the stimulation position; and
the stimulation control circuit operating to distinguish artifact signals in the electrical activity signals unrelated to the stimulation based from nerve signals in the electrical activity signals related to the stimulation based on imputed location of an effective stimulation points individually consistent with the artifact signals and nerve signals; and
wherein the stimulation control circuit uses the identified artifacts signals to display the effective stimulation points for the nerve signals distinguished from effective stimulation points of the artifacts signals; the method comprising;
(a) deducing a set of effective stimulation points associated with the electrical activity signals;
(b) distinguishing artifacts signals in the electrical activity signals based on imputed location of effective simulation points for the artifacts signals; and
(c) use the distinguish artifact signals to display effective stimulation points of the nerve signals distinguished from the artifacts signals.

18. A method of employing a neural stimulation device having:
a set of implantable electrodes positionable along a nerve;
a stimulation power source communicating at least one electrode of the set of implantable electrodes and producing a nerve stimulation signal;
a stimulation control circuit controlling the stimulation power source to selectively apply stimulation to at least one stimulation electrode selected from the implantable electrodes at a stimulation power, a position, and a time and communicating with electrodes of the set of implantable electrodes other than the at least one stimulating electrode for monitoring electrical activity signals at multiple monitoring electrodes selected from the implantable electrodes and displaced from the stimulation position, the method comprising:
(a) deducing an effective stimulation point of the stimulation based on the electrical activity signals at the multiple monitoring electrodes; and
(b) based on the deduced effective stimulation point, modify at least one of the power of the stimulation and position of the stimulation electrode.
